(19) United States
(12) Patent Application Publication
(10) Pub. No.: US 2016/0263407 A1
(43) Pub. Date: Sep. 15, 2016

(54) Method For Providing Maximum Malodor and Perspiration Control

(71) Applicant: The Procter & Gamble Company, Cincinnati, OH (US)

(72) Inventor: James Robert SCHWARTZ, West Chester, OH (US)

(21) Appl. No.: 15/162,066

(22) Filed: May 23, 2016

Related U.S. Application Data
(63) Continuation of application No. 13/028,873, filed on Feb. 16, 2011, now abandoned.
(60) Provisional application No. 61/305,131, filed on Feb. 16, 2010.

(51) Int. Cl. A61Q 15/00; A61K 8/58

(52) U.S. Cl. CPC A61Q 15/00; A61K 8/58

(57) ABSTRACT

A package including an intimate cleansing product, a deodorant composition, and in some cases a razor is disclosed herein. The deodorant composition includes an anti-irritation agent. The intimate cleansing product, deodorant composition and razor are co-packaged together. Or, the intimate cleansing product and the deodorant composition are co-packaged together.

### FIELD OF THE INVENTION

The present invention is related to a line of consumer products for providing odor and perspiration control.

### BACKGROUND OF THE INVENTION

Antiperspirant compositions are designed to provide effective perspiration control while also being an acceptable composition so desired by the consumer. Antiperspirant composition also seed to provide odor control. The odor control mechanisms of antiperspirants derive from controlling the perspiration and providing perfumes for masking purposes. For some consumers, these mechanisms do not provide sufficient odor benefit, as the odor overwhelms the capabilities of the antiperspirant composition. Thus, there is a need for a secondary product to provide enhanced odor control.

### SUMMARY OF THE INVENTION

The present invention relates to a method for treating malodor on an intimate area comprising: applying an intimate cleansing product to the intimate area; rinsing the intimate cleansing product from the intimate area; and applying an antiperspirant to the intimate area; wherein the intimate cleansing product comprises a malodor active.

### DETAILED DESCRIPTION OF THE INVENTION

While the specification concludes with the claims particularly pointing and distinctly claiming the invention, it is believed that the present invention will be better understood from the following description.

As used herein, “comprising” means that other steps and/or ingredients can be added. This term encompasses the terms “consisting of”0 and “consisting essentially of”. The phrase “consisting essentially of” means that the invention may include additional steps and/or ingredients, but only if the additional ingredients and/or steps do not materially alter the basic and novel characteristics of the claimed invention or methods.

All percentages, parts and ratios are based upon the total weight of the topical compositions of the present invention and all measurements made are at 25° C., unless otherwise specified. All such weights as they pertain to listed ingredients are based on the active level and, therefore; do not include carriers or by-products that may be included in commercially available materials, unless otherwise specified.

The present invention involves a method for treating malodor on an intimate area comprising applying an intimate cleansing product to the intimate area, rinsing the intimate cleansing product from the intimate area, applying an antiperspirant or deodorant to the intimate area, wherein the intimate cleansing product comprises a malodor active. In one embodiment, the applying and rinsing step occurs before a sleep cycle.

### Intimate Cleansing Product

The intimate cleansing product described herein may include a variety of personal care products including, but not limited to, body wash, body sprays, lotions, creams, and the like. Depending on the form, the intimate cleansing product may be applied by direct application, placement on a washing implement, spraying, and the like. Washing implements include sponges, towels, bath puffs, brushes, and the like. Without wishing to be bound by theory, it is believed that a washing implement provides an additional scrubbing mechanism allowing further penetration of the intimate cleansing product into the pores and/or follicles of the skin.

The intimate cleansing product is suitable for application to an intimate area. Intimate areas of the present invention include the underarm, crotch, feet, intergluteal cleft, and the like. In one embodiment, the intimate cleansing product of this invention is utilized on the underarm. The application of the intimate cleansing product may take place during a shower or while taking a bath. It is also contemplated that the application of the intimate cleansing product may take place outside of the shower or bath, by applying and rinsing utilizing a wash sink.

It is contemplated that the intimate cleansing product of the present invention is removed after application. Typical removing methods including rinsing, in either the shower or bath, and/or utilizing a washing implement. One of ordinary skill is readily capable of determining an effective method of removing the intimate cleansing product from an intimate area.

In one embodiment, the intimate cleansing product comprises a malodor active. The malodor active of the present invention is capable of providing an antimicrobial benefit. Such malodor actives are capable of destroying microbes, preventing the development of microbes or preventing the pathogenic action of microbes. A safe and effective amount of a malodor active may be added to the intimate cleansing product, at from about 0.001% to about 10%, or from about 0.01% to about 5%, or from about 0.05% to about 2%, or from about 0.1% to about 1%, or from about 0.3% to about 0.7%, or about 0.5% by weight of, e.g. the cleansing product.

Examples of malodor actives include β-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, zinc pyrithione and clotrimazole.

### Zinc Salt

The composition of the present invention optionally includes an effective amount of a zinc salt.

Preferred embodiments of the present invention include an effective amount of a zinc salt having an aqueous solubility within the composition of less than about 25%, by weight, at 25° C., more preferably less than about 20%; more preferably less than about 15%.

Preferred embodiments of the present invention include from 0.001% to 10% of a zinc salt; more preferably from 0.01% to 5%; more preferably still from 0.1% to 3%.

In a preferred embodiment, the zinc salt has an average particle size of from 100 nm to 30 μm.

Examples of zinc salts useful in certain embodiments of the present invention include the following: Zinc aluminate, Zinc carbonate, Zinc oxide and materials containing zinc oxide (i.e., calamine), Zinc phosphates (i.e., orthophosphate and pyrophosphate), Zinc selenide, Zinc sulfide, Zinc silicates (i.e., ortho- and meta-zinc silicates), Zinc silicofluoride, Zinc Borate, Zinc hydroxide and hydroxy sulfate, zinc-containing layered materials and combinations thereof.

In embodiments having an anti-irritation agent and a zinc salt, the ratio of zinc salt to anti-irritation agent is preferably from 5:100 to 5:1; more preferably from about 2:10 to 3:1; more preferably still from 1:2 to 2:1.

### Antiperspirant/Deodorant Compositions

The antiperspirant and deodorant substances for use with the intimate cleansing product of the present invention are compositions which are intended for topical application to the underarm or other suitable areas of the skin. The terms “antiperspirant active” and “deodorant active” specifically refers to topical materials which can prevent or eliminate malodors and/or perspiration wetness.

### Antiperspirant Active

The antiperspirant compositions for use in the present invention comprise antiperspirant active suitable for application to human skin. The antiperspirant active may be solubilized in the antiperspirant compositions or may be suspended as an undissolved or precipitated solid. The concentration of the antiperspirant active in the antiperspirant compositions should be sufficient to provide the desired odor and wetness control from the antiperspirant composition selected.

The antiperspirant compositions of the present invention can be formulated as any known or otherwise effective product form for providing topical application of antiperspirant or deodorant active to the desired area of the skin. Non-limiting examples of such product forms include liquids (e.g., aerosols, pump sprays, roll-ons), solids (e.g., gel solids, invisible solids, wax solid sticks), semi-solids (e.g. creams, soft solids, lotions), and the like. For example, the antiperspirant compositions of the present invention may be semi-solids or solids.

The antiperspirant products are generally stored in and dispensed from a suitable package or applicator device. For example, the package or applicator device may be a cream dispenser with perforated application domes, etc. These packages should be sufficiently closed to prevent excessive loss of volatiles prior to application.

The antiperspirant compositions described herein comprise antiperspirant active at concentrations of from about 0.5% to about 60%, preferably from about 0.5% to about 50%, more preferably from about 5% to about 35%, by weight of the selected antiperspirant composition. All such weight percentages are calculated on an anhydrous metal salt basis exclusive of water and any complexing agents such as glycine, glycine salts, or other complexing agents.

The antiperspirant active for use in the antiperspirant compositions described herein include any compound, composition or other material having antiperspirant activity. Preferred antiperspirant actives include the astringent metallic salts, especially the inorganic and organic salts of aluminum, zirconium and zinc, as well as mixtures thereof. Particularly preferred are the aluminum and zirconium salts, such as aluminum halides, aluminum hydroxyhalides, zirconyl oxyhalides, zirconyl hydroxyhalides, and mixtures thereof.

The antiperspirant active may be formulated as particulate solids in the form of dispersed solid particles having a preferred average particle size or diameter of less than about 100 μm, more preferably from about 2 μm to about 50 μm, even more preferably from about 0.4 μm to about 40 μm.

The antiperspirant compositions described herein may comprise solubilized antiperspirant active, preferably solubilized antiperspirant active in an anhydrous system. The concentration of solubilized antiperspirant active in the antiperspirant compositions preferably ranges from about 0.1% to 35%/, more preferably from about 0.5% to about 25%, even more preferably from about 1% to about 17%, even more preferably from about 6% to about 17%, by weight of the selected antiperspirant composition (weight percentages calculated on an anhydrous metal salt basis exclusive of water and any complexing agents such as glycine, glycine salts, or other complexing agents).

### Deodorant Active

The deodorant compositions for use in the applicator of the present invention comprise deodorant active at concentrations ranging from about 0.001% to about 50%, preferably from about 0.01% to about 20%, more preferably from about 0.1% to about 10%, even more preferably from about 0.1% to about 5%, by weight of the selected deodorant composition. These deodorant actives can include any known or otherwise safe and effective deodorant active suitable for topical application to human skin.

Deodorant actives suitable for use in the deodorant compositions described herein include any topical material that is known for or is otherwise effective in preventing or eliminating malodor associated with perspiration. These deodorant actives are typically antimicrobial agents (e.g., bacteriocides, fungicides), malodor-absorbing material, or combinations thereof.

Preferred deodorant actives are antimicrobial agents, non-limiting examples of which include cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichlorio-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, farnesol, and combinations thereof.

Other deodorant actives include odor-absorbing materials such as carbonate and bicarbonate salts, including alkali metal carbonates and bicarbonates, ammonium and tetraalkylammonium. Preferred are sodium and potassium salts of such odor-absorbing materials.

The deodorant compositions of the present invention can be formulated as any known or otherwise effective product form for providing topical application of antiperspirant or deodorant active to the desired area of the skin. Non-limiting examples of such product forms include liquids (e.g., aerosols, pump sprays, roll-ons), solids (e.g., gel solids, invisible solids, wax solid sticks), semi-solids (e.g. creams, soft solids, lotions), and the like. For example, the antiperspirant compositions of the present invention may be semi-solids or solids.

The deodorant products are generally stored in and dispensed from a suitable package or applicator device. For example, the package or applicator device may be a cream dispenser with perforated application domes, etc. These packages should be sufficiently closed to prevent excessive loss of volatiles prior to application.

### Method of Use

It is contemplated that the intimate cleansing product and the antiperspirant or deodorant will be used sequentially, within an hour of each other. For example, after the intimate cleansing product is applied and rinsed, the antiperspirant or deodorant is applied within an hour of the completion of the shower. It is also contemplated that a drying step may occur between the rinsing step and the application step of the antiperspirant or deodorant. Such drying step can be accomplished by air drying (either hot air or room-temperature air) or by drying with a towel or alternate substrate. One of ordinary skill would readily know mechanisms for drying an intimate area.

Further improved efficacy can be accomplished by performing this invention before a sleep cycle, though use after a sleep cycle is also contemplated. Generally, a sleep cycle includes a rest period of time of at least about 1 hour, at least about 2 hours, at least about 4 hours or at least about 8 hours. Typically, the sleep cycle begins after sunset but before sunrise. However, it is contemplated that individuals may utilize non-traditional sleep cycles.

Without being limited to the above treatment regimens, the present inventors have found that such treatment regimens may help to treat hyperhidrosis by achieving effective antiperspirant reduction or inhibition over an extended period.

### Co-Packaging

The products of the present invention may be packaged individually in separate containers or they may be packaged together in a unitary form to be sold and bought together. Additionally, these products may also be packaged with a razor, representing a complete regiment for a consumer. Written and/or graphic instructions may be included in the separate packages or in the unitary packages to instruct a consumer when and how to use the product. Additionally, the product or products may be packaged in a secondary package wherein an outer container embodies the product disclosed therein.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited herein are, in the relevant part, incorporated herein by reference; the citation of any document is not to be construed as an admission that it is prior art with respect to the present invention. To the extent that any meaning or definition of a term or in this written document conflicts with any meaning or definition in a document incorporated by reference, the meaning or definition assigned to the term in this written document shall govern.

Except as otherwise noted, the articles “a”, “an”, and “the” mean “one or more”.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1-18. (canceled)

19. A package comprising:
a razor;
a deodorant composition comprising a deodorant active and an anti-irritation agent; and
an intimate cleansing product, wherein the razor, deodorant composition and intimate cleansing product are co-packaged products and are provided with instructions for use on the crotch and/or intergluteal cleft of a user.

20. The package of claim 19, wherein the deodorant composition comprises a zinc salt.

21. The package of claim 20, wherein a ratio of anti-irritation agent and zinc salt is from about 2 to 10 to about 3 to 1.

22. The package of claim 20, wherein the zinc salt consists of zinc oxide.

23. The package of claim 20, wherein the zinc salt has an aqueous solubility within the deodorant composition of less than about 20 percent by weight at 25 degrees Centigrade.

24. The package of claim 20, wherein the zinc salt is present in the deodorant composition at from 0.1 percent to 3 percent.

25. The package of claim 19, wherein the deodorant active comprises carbonate and/or bicarbonate salts.

26. The package of claim 19, wherein the deodorant composition is provided in the form of a solid stick.

27. The package of claim 19, wherein the deodorant composition is provided in the form of a roll-on.

28. The package of claim 19, wherein the deodorant composition is provided in the form of a cream.

29. The package of claim 19, wherein the intimate cleansing product comprises zinc pyrithione.

30. The package of claim 19, wherein the deodorant composition comprises an antimicrobial agent.

31. The package of claim 19 further comprising a washing implement comprising at least one of a sponge, towel, bath puff, and brush.

32. A package comprising:
a deodorant composition comprising an anti-irritation agent;
an intimate cleansing product, wherein deodorant composition and intimate cleansing product are co-packaged products and are provided with instructions for use on the crotch and/or intergluteal cleft of a user.

33. The package of claim 32, wherein the deodorant composition comprises zinc oxide.

34. The package of claim 33, wherein the zinc oxide has an average particle size of 100 nanometers.

35. The package of claim 33, wherein the zinc oxide has an average particle size of 30 micrometers.

36. The package of claim 33, wherein the zinc oxide has an average particle size of between 100 nanometers to 30 micrometers.

37. The package of claim 33, wherein deodorant composition comprises from 0.01 percent to 5 percent of zinc oxide.

38. The package of claim 32, wherein the deodorant composition is provided in the form of a solid stick.
